(19) United States
(12) Patent Application Publication
(10) Pub. No.: US 2023/0016997 A1
(43) Pub. Date: Jan. 19, 2023

(54) IMAGE ACQUISITION MEDICAL DEVICE AND MEDICAL SYSTEM

(71) Applicant: TERUMO KABUSHIKI KAISHA, Tokyo (JP)

(72) Inventors: Yasunori YAMASHITA, Fujinomiya-shi (JP); Tomoji MARUYAMA, Hadano-shi (JP)

(73) Assignee: TERUMO KABUSHIKI KAISHA, Tokyo (JP)

(21) Appl. No.: 17/937,093

(22) Filed: Sep. 30, 2022

Related U.S. Application Data
(63) Continuation of application No. PCT/JP2021/008622, filed on Mar. 5, 2021.

(30) Foreign Application Priority Data
Mar. 31, 2020 (JP) 2020-062154

(51) Int. Cl. G06T 7/00; G06T 7/62; G06T 7/70; G06F 3/14; A61M 25/09

(52) U.S. Cl. CPC G06T 7/0012; G06T 7/62; G06T 7/70; G06F 3/14; A61M 25/0905; G06T 2207/10072; G06T 2207/30021

(57) ABSTRACT

The disclosed image acquisition medical device and medical system make it possible to easily grasp an orientation of a distal end portion of the medical device based on an angiographic image and a tomographic image. The image acquisition medical device includes a flexible body portion that extends in an axial direction; an image sensor that is disposed in the body portion and that is configured to acquire an image of a hollow organ; and a contrast unit that protrudes toward a distal end side of the body portion and that makes an orientation of a distal end portion of the body portion visually recognizable in an angiographic image. Relative positions of the image sensor and the contrast unit in an axial rotation direction are fixed.

### CROSS-REFERENCES TO RELATED APPLICATIONS

This application is a continuation of International Patent Application No. PCT/JP2021/008622 filed on Mar. 5, 2021, which claims priority to Japanese Patent Application No. 2020-062154 filed on Mar. 31, 2020, the entire content of both of which is incorporated herein by reference.

### TECHNOLOGICAL FIELD

The present invention generally relates to an image acquisition medical device and a medical system.

### BACKGROUND DISCUSSION

In treatment of a stenosed portion or an occluded portion that occurs in a hollow organ such as a blood vessel and a vascular channel, in order to observe behaviors thereof or a state after the treatment, an image of the hollow organ is acquired using an inspection wave such as an ultrasound or light. In order to acquire the image of the hollow organ at a relatively low cost, a medical device equipped with an image wire including an image sensor is proposed (see JP-B-4878823).

The image wire includes a wire portion that extends in an axial direction and the image sensor that is disposed on the wire portion and that acquires the image of the hollow organ. The image wire is connected to a control unit including a display unit via a cable and the like, and transmits and receives a signal to and from the control unit.

An operator such as a doctor can obtain a tomographic image of the hollow organ such as a blood vessel by using the image wire of JP-B-4878823. The operator acquires an angiographic image (mainly a contrast image of the image wire in an extending direction of the blood vessel) when moving the image wire of JP-B-4878823 in the blood vessel.

However, in the image wire, a body portion of the image wire is not displayed in the tomographic image. Therefore, there is a problem that the operator cannot confirm a relative orientation between the angiographic image and the tomographic image. Such a problem may also occur in an electronic scanning type image diagnostic catheter, in which a guidewire is inserted inside the image sensor, and the like.

### SUMMARY

The image acquisition medical device and medical system disclosed here make it possible to easily grasp or understand an orientation of a distal end portion based on an angiographic image and a tomographic image.

An image acquisition medical device of the present disclosure includes a flexible body portion that extends in an axial direction; an image sensor that is disposed in the body portion and that is configured to acquire an image of a hollow organ such as a blood vessel; and a contrast unit that protrudes toward a distal end side of the body portion and that makes an orientation of a distal end portion of the body portion visually recognizable in an angiographic image. Relative positions of the image sensor and the contrast unit in an axial rotation direction are fixed.

A medical system of the present disclosure includes an image acquisition medical device comprised of a flexible body portion and an image sensor disposed in the flexible body portion and configured to acquire an angiographic image and a tomographic image of a hollow organ; a display device that is configured to display an angiographic image and a tomographic image acquired by an image acquisition medical device that is insertable into a hollow organ; and a control device that is configured to control a display content of the display device. The control device controls the display content to display an orientation of a distal end portion of the image acquisition medical device in the tomographic image.

According to the image acquisition medical device and the medical system of the present disclosure, the operator can easily grasp a relative orientation between the angiographic image and the tomographic image by visually recognizing the orientation of the contrast unit displayed in the angiographic image and the tomographic image and the distal end portion of the image acquisition medical device displayed on the tomographic image.

Another aspect of the disclosure involves a method comprising viewing, on a display, tomographic images and angiographic images of a distal end portion of an image wire comprised of: a flexible body portion that is positioned in a hollow organ; an image sensor disposed in the flexible body portion and configured to acquire the tomographic and angiographic images of the hollow organ; and a contrast unit connected to a distal end of the flexible body portion and protruding in a distal direction from the distal end of the flexible body portion, with relative positions of the image sensor and the contrast unit in an axial rotation direction being fixed. The method additionally includes rotating the image wire that is positioned in a hollow organ so that an orientation of a distal end of the contrast unit changes in the tomographic images and the angiographic images on the display, and determining the orientation of a distal end portion of the flexible body portion by viewing a change in the orientation of the distal end of the contrast unit in the tomographic images and the angiographic images displayed on the display.

### BRIEF DESCRIPTION OF THE DRAWINGS

FIG. 1 is a diagram schematically illustrating a medical system that includes an image acquisition medical device;

FIG. 2 is a diagram illustrating an example of a tomographic image acquired by an image sensor;

FIGS. 3A and 3B are diagrams illustrating an example of an angiographic image acquired during a procedure using the image acquisition medical device;

FIG. 4 is a diagram illustrating an example of the tomographic image acquired by the image sensor;

FIGS. 5A and 5B are diagrams illustrating an example of the angiographic image acquired during the procedure using the image acquisition medical device;

FIG. 6 is a diagram illustrating an example of the tomographic image acquired by the image sensor;

FIGS. 7A and 7B are diagrams illustrating an example of the angiographic image acquired during the procedure using the image acquisition medical device; and

FIG. 8 is a diagram schematically illustrating a medical system according to a modification.

### DETAILED DESCRIPTION

Set forth below with reference to the accompanying drawings is a detailed description of embodiments of an image acquisition medical device and a medical system representing examples of the image acquisition medical device and medical system disclosed here, but a technical scope of the invention should be defined based on the description of the claims, and is not limited to the following embodiment.

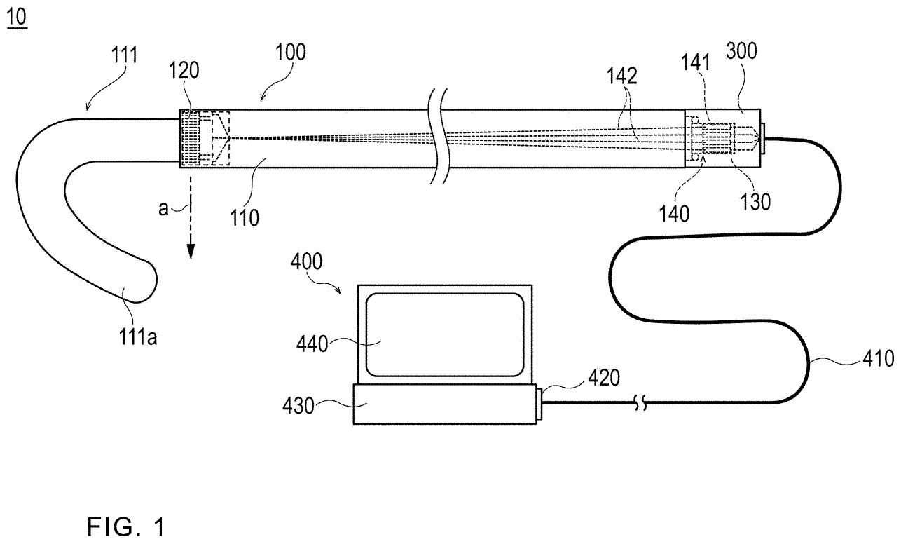

In the following description, a proximal side (upper side in FIG. 1) of an image wire 100 is referred to as a “proximal end”, and a side (lower side in FIG. 1) inserted into a body cavity is referred to as a “distal end”. A direction from the proximal end to the distal end of the image wire 100 is referred to as an “axial direction”. In order to facilitate understanding by the description of the drawings, the image wire 100 is schematically illustrated such that a length thereof is reduced and a thickness thereof is exaggerated, and a ratio of the length to the thickness is different from an actual ratio.

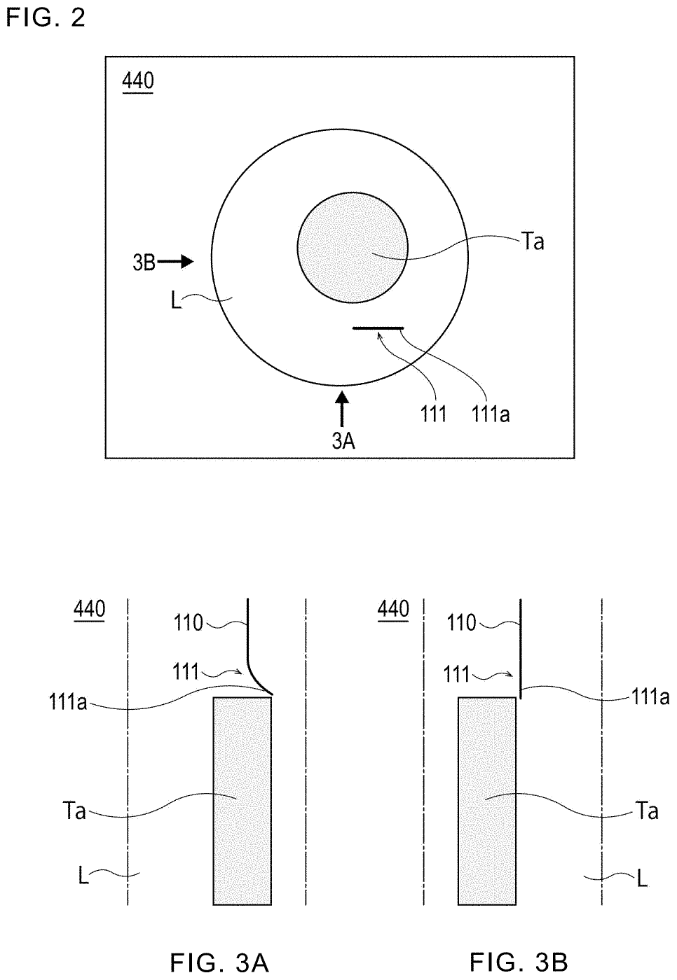

FIG. 1 is a diagram illustrating a medical system 10 that includes the image wire 100, and FIGS. 2 to 7B are diagrams illustrating examples of a tomographic image and an angiographic image displayed on a monitor 440.

(Image Wire 100)

In the present embodiment, an example of an image acquisition medical device is the image wire 100.

As illustrated in FIG. 1, the image wire 100 includes a flexible body portion 110 that extends in the axial direction, an image sensor 120 that is disposed in the body portion 110 and that acquires an image of a hollow organ, and a contrast unit 111 that protrudes toward a distal end side of the body portion 110 (in the distal direction) and that makes an orientation of the distal end portion of the body portion 110 visually recognizable in an angiographic image and a tomographic image acquired by the image sensor 120. Relative positions of the image sensor 120 and the contrast unit 111 in an axial rotation direction are fixed.

The body portion 110 is implemented by a flexible wire. A constituent material from which the body portion 110 may be fabricated is not particularly limited as long as the body portion 110 is flexible, and for example, various metal materials such as stainless steel, a cobalt-based alloy, and a superelastic alloy can be used.

The contrast unit 111 is implemented by a metal wire material that protrudes in a direction intersecting the axial direction of the body portion 110. The contrast unit 111 may be implemented by the same material as the materials exemplified as the constituent material of the body portion 110. The contrast unit 111 may be implemented by, for example, a wire material having the same configuration as a known guidewire in a medical field. The contrast unit 111 and the body portion 110 may be connected by, for example, welding. As illustrated in FIG. 1, the contrast unit 111 may have a diameter (outer diameter) smaller than a diameter (outer diameter) of the body portion 110.

The image wire 100 may be implemented such that the contrast unit 111 disposed on the distal end side is highly flexible and the body portion 110 located on the proximal end side of the contrast unit 111 has relatively high rigidity. Accordingly, the image wire 100 can achieve both flexibility of the distal end and excellent operability (pushing property, torque transmitting property, and the like).

As illustrated in FIG. 1, the contrast unit 111 is shaped to be curved in a direction intersecting the axial direction of the body portion 110 in a natural state where no external force is applied. As shown in FIG. 1, the contrast unit 111 may be J-shaped or U-shaped. The contrast unit 111 may be configured so that a portion of the contrast unit 111 located immediately adjacent the distal end of the body portion 110 (immediately adjacent the image sensor 120) extends distally away from the distal end of the body portion 110 and the image sensor 120 along the center axis of the body portion 110. As illustrated in FIG. 1, a distal end 111a of the contrast unit 111 is disposed at a position separated (spaced) from an axial center of the body portion 110 (radially outwardly spaced from an axial center of the body portion 110) in any side view of the image wire 100.

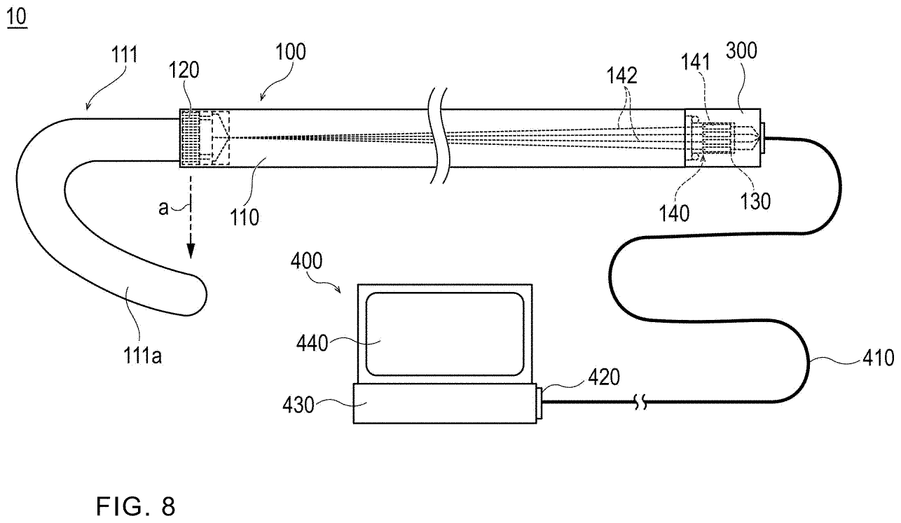

For example, in the natural state of the contrast unit 111 illustrated in FIG. 1 (a state in which no force is applied to the contrast unit), the distal end 111a of the contrast unit 111 may be disposed at a position that does not overlap the image sensor 120 at the position separated outward in the radiation direction (radially outward) from the axial center of the body portion 110. Stated differently, a plane perpendicular to the axial center of the body portion and passing through the image sensor 120 does not intersect the distal end 111a of the contrast unit 111. Instead, the distal end 111a of the contrast unit 111 is distal of such plane. However, as in the modification illustrated in FIG. 8, the distal end 111a of the contrast unit 111 may also be disposed at a position that overlaps the image sensor 120 at the position separated outward in the radiation direction (radially outward) from the axial center of the body portion 110 in the natural state, for example. That is, a plane perpendicular to the axial center of the body portion and passing through the image sensor 120 intersects the distal end 111a of the contrast unit 111. By disposing the distal end 111a of the contrast unit 111 in this way, the distal end 111a of the contrast unit 111 can be displayed when the tomographic image is acquired by an inspection wave (illustrated by an arrow a in FIG. 1) radiated from the image sensor 120.

A shape of the contrast unit 111 is not particularly limited as long as the contrast unit protrudes in a direction intersecting the axial direction of the body portion 110. That is, contrast unit 111 is configured in a way that deviates or extends away from the axial direction or central axis of the body portion 110. For example, the contrast unit 111 may have a shape bent in a straight line at an obtuse angle or an acute angle with respect to the body portion 110. The contrast unit 111 is thus configured so that it is other than a straight-line continuation or extension of the body portion 110.

The image sensor 120 is disposed on the distal end of the body portion 110.

The image sensor 120 is implemented by a phased array in which a plurality of sensor elements are arranged in a ring shape. By using such a phased array type image sensor 120, a tomographic image of the hollow organ can be acquired at once over a wide range in a circumferential direction without rotating the image sensor 120. The sensor elements are not particularly limited, and include an intravascular ultrasound (IVUS) sensor element, an optical coherence tomography (OCT) sensor element, and the like. A type, arrangement, and the number of the sensor elements used in the image sensor 120 are not particularly limited.

As illustrated in FIG. 1, the image wire 100 includes an electrical contact portion 140 disposed on the proximal end portion 130 of the body portion 110, and a connector 300 that is insertable into and removable from the proximal end portion 130 of the body portion 110 and is electrically connected to the electrical contact portion 140.

The electrical contact portion 140 includes a plurality of terminal portions 141. Adjacent terminal portions 141 are electrically insulated. The sensor elements of the image sensor 120 and the terminal portions 141 are arranged in pairs. A pair of sensor element and terminal portion 141 are electrically connected to each other via a signal line 142.

The connector 300 electrically connects the electrical contact portion 140 and a control device 400 via a cable 410. By connecting the control device 400 to the image sensor 120 via the connector 300 and the cable 410, the control device 400 can transmit and receive an electric signal to and from the image sensor 120.

(Medical System 10)

As illustrated in FIG. 1, the medical system 10 includes the image wire 100 and the control device 400.

As described above, the control device 400 is connected to the image wire 100 via the cable 410.

The connector 300 connected to the image wire 100 is attached to one end of the cable 410, and another connector 420 connected to the control device 400 is attached to the other end of the cable 410.

The control device 400 includes a control unit 430 and a monitor (corresponding to a display device or a display) 440.

The control unit 430 mainly includes a CPU, a memory, and an input and output unit, and controls the entire medical system 10. For example, the control unit 430 outputs a control signal for emitting the inspection wave to the image sensor 120, inputs a detection signal from the image sensor 120, and acquires image data based on the detection signal.

The control unit 430 displays information (video) based on the acquired image data on the monitor 440. In the present embodiment, the control unit 430 can display an angiographic image and a tomographic image, which will be described later, on the monitor 440.

Next, a function of the contrast unit 111 will be described with reference to FIGS. 2 to 7.

FIGS. 2, 4, and 6 illustrate examples of the tomographic image displayed on the monitor 440. FIGS. 3A, 3B, 5A, 5B, 7A and 7B illustrate examples of the angiographic image displayed on the monitor 440. Here, examples in which the angiographic image is displayed on the monitor 440 are illustrated, but the angiographic image may be displayed on a display device other than the monitor 440. The control device 400 controls a display content of the monitor 440 to display an orientation of a distal end of the image wire 100 (an orientation of the distal end 111a of the contrast unit 111) on the tomographic image. In the medical system 10, the orientation of the distal end 111a of the contrast unit 111 in the tomographic image can be indicated by a virtual point, a line, an arrow, or the like on the tomographic image.

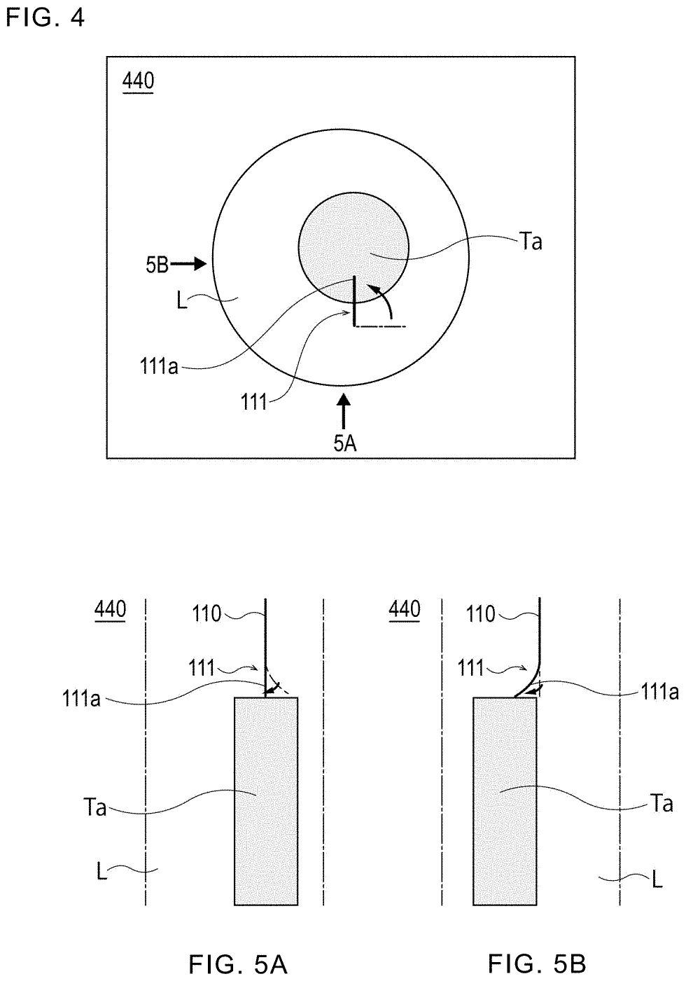

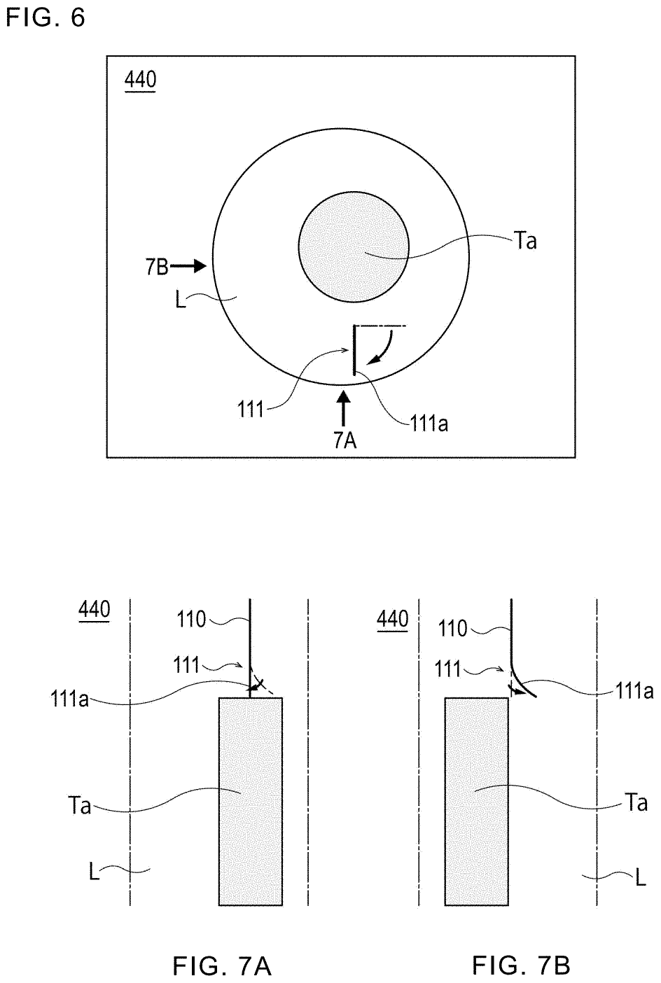

FIG. 3A illustrates an angiographic image acquired from a direction of an arrow 3A (direction at left front inclination 45°) in FIG. 2. FIG. 3B illustrates an angiographic image acquired from a direction of an arrow 3B (direction at right front inclination 45°) in FIG. 2. FIG. 5A illustrates an angiographic image acquired from a direction of an arrow 5A (direction at left front inclination 45°) in FIG. 4. FIG. 5B illustrates an angiographic image acquired from a direction of an arrow 5B (direction at right front inclination 45°) in FIG. 4. FIG. 7A illustrates an angiographic image acquired from a direction of an arrow 7A (direction at left front inclination 45°) in FIG. 6. FIG. 7B illustrates an angiographic image acquired from a direction of an arrow 7B (direction at right front inclination 45°) in FIG. 6.

When performing a predetermined treatment in a hollow organ L (for example, a blood vessel), the operator may determine a moving direction, an orientation, or the like of a second guide wire (for example, a guide wire for guiding movement of a catheter device for treatment) based on the tomographic image and the angiographic image acquired by the image wire 100.

FIG. 2 illustrates a state in which the image wire 100 is delivered to the vicinity of a predetermined target site (for example, a true cavity) Ta of the hollow organ L.

The operator grasps the orientation of the distal end portion of the body portion 110 of the image wire 100 based on the tomographic image and the angiographic image. Specifically, the operator confirms the orientation of the distal end 111a of the contrast unit 111 by the tomographic image of FIG. 2 and the angiographic images of FIGS. 3A and 3B. For example, when the operator rotates the image wire 100 clockwise, the orientation of the distal end 111a of the contrast unit 111 changes in the tomographic image of FIG. 4 and the angiographic images of FIGS. 5A and 5B. For example, when the operator rotates the image wire 100 counterclockwise, the orientation of the distal end 111a of the contrast unit 111 changes in the tomographic image of FIG. 6 and the angiographic images of FIGS. 7A and 7B. For example, in each angiographic image, the operator can easily grasp the orientation of the distal end portion of the body portion 110 of the image wire 100 by visually recognizing the tomographic image while visually recognizing a state in which the distal end 111a of the contrast unit 111 is disposed at a position farthest from the body portion 110 (a state in which the distal end 111a is displayed most long such as FIG. 7B, compared to FIG. 7A in which the distal end 111a is displayed shorter (shortest)). Accordingly, the control device 400 can grasp in which direction of the tomographic image the distal end 111a of the contrast unit 111 is oriented, even when the contrast unit 111 is not displayed in the tomographic image.

The medical system 10 allows the control device 400 to recognize the state in which the distal end 111a of the contrast unit 111 is disposed at the position farthest from the body portion 110, for example, toward the right with respect to the axial direction of the body portion 110 on an angiographic image plane (by an operation such as an input operation by the operator), and matches the rotation orientation of the tomographic image at that time such that the distal end 111a of the contrast unit 111 faces the right, thereby making it possible to match the left-right orientation of the image sensor 120 on the angiographic image plane with respect to the axial direction with the left-right orientation of the tomographic image.

The operator acquires tomographic images and angiographic images before and after the rotation while appropriately rotating the image wire 100 as described above. The operator can easily grasp the orientation of the distal end portion of the body portion 110 by confirming the orientation of the distal end 111a of the contrast unit 111 displayed in the acquired tomographic images and the angiographic images. The operator can confirm a relative positional relation between the distal end portion of the body portion 110 and the target site Ta by grasping the orientation of the distal end portion of the body portion 110. The operator can easily guide the second guide wire to the target site Ta by operating the second guide wire within the hollow organ L based on the relative positional relation between the distal end portion of the body portion 110 and the target site Ta.

Although the image acquisition medical device and the medical system are described above through the embodiment, the image acquisition medical device and the medical system are not limited to the content described in the specification, and may be appropriately changed based on the description of the claims.

The image acquisition medical device may be implemented by, for example, an electronic scanning type image acquisition (image diagnosis) catheter device.

The detailed description above describes embodiments of an image acquisition medical device and the medical system representing examples of the inventive image acquisition medical device and medical system disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An image acquisition medical device comprising:
a flexible body portion that extends in an axial direction;
an image sensor that is disposed in the flexible body portion and that is configured to acquire an image of a hollow organ;
a contrast unit that protrudes toward a distal end side of the flexible body portion and that makes an orientation of a distal end portion of the flexible body portion visually recognizable in an angiographic image; and
relative positions of the image sensor and the contrast unit in an axial rotation direction are fixed.

2. The image acquisition medical device according to claim 1, wherein the contrast unit is a metal wire that protrudes in a direction intersecting the axial direction of the flexible body portion.

3. The image acquisition medical device according to claim 2, wherein a distal end of the contrast unit is disposed at a position that overlaps the image sensor at a position separated outward in a radial outward direction from an axial center of the flexible body portion.

4. The image acquisition medical device according to claim 1, wherein the flexible body portion is a flexible wire.

5. The image acquisition medical device according to claim 1, wherein the image sensor is a phased array image sensor in which a plurality of sensor elements are arranged in a ring shape.

6. The image acquisition medical device according to claim 1, wherein the contrast unit includes a curved portion as seen in a side view of the image acquisition medical device.

7. The image acquisition medical device according to claim 1, wherein the contrast unit is J-shaped as seen in a side view of the image acquisition medical device.

8. The image acquisition medical device according to claim 1, wherein the flexible body portion includes a distal end, the contrast unit including a first portion positioned immediately adjacent the distal end of the flexible body portion, the first portion of the contrast unit extending in a distal direction along a center axis of the flexible body portion and away from the distal end of the flexible body portion.

9. The image acquisition medical device according to claim 8, wherein the contrast unit includes a second portion that is spaced from the distal end of the flexible body portion, the second portion of the contrast unit extending radially away from the center axis of the body portion.

10. The image acquisition medical device according to claim 1, wherein the flexible body portion possesses an outer diameter and the contrast unit possesses an outer diameter, the outer diameter of a distal end of the flexible body portion that is connected to a proximal end of the contrast unit being larger than an outer diameter of the proximal end of the contrast unit.

11. A medical system comprising:
an image acquisition medical device comprised of a flexible body portion and an image sensor, the image sensor being disposed in the flexible body portion;
a display device configured to display an angiographic image and a tomographic image acquired by the image acquisition medical device that is insertable into a hollow organ;
a control device connected to the display device and configured to control a display content of the display device; and
the control device being configured to control the display content to display an orientation of a distal end portion of the image acquisition medical device in the tomographic image.

12. The medical system according to claim 11, wherein the control device is configured to control the display content such that a left-right orientation of the image acquisition medical device on an angiographic image plane with respect to an axial direction and a left-right orientation of the tomographic image are matched and displayed on the display device.

13. The medical system according to claim 11, wherein the distal end portion of the image acquisition medical device is a contrast unit that protrudes toward a distal end side of the flexible body portion.

14. The medical system according to claim 13, wherein the contrast unit is a metal wire that protrudes in a direction intersecting an axial direction of the flexible body portion.

15. The medical system according to claim 13, wherein the contrast unit includes a curved portion as seen in a side view of the image acquisition medical device.

16. The medical system according to claim 13, wherein the flexible body portion possesses an outer diameter and the contrast unit possesses an outer diameter, the outer diameter of a distal end of the flexible body portion that is connected to a proximal end of the contrast unit being larger than an outer diameter of the proximal end of the contrast unit.

17. A method comprising:
viewing, on a display, tomographic images and angiographic images of a distal end portion of an image wire comprised of: a flexible body portion that is positioned in a hollow organ; an image sensor disposed in the flexible body portion and configured to acquire the tomographic and angiographic images of the hollow organ; and a contrast unit connected to a distal end of the flexible body portion and protruding in a distal direction from the distal end of the flexible body portion, with relative positions of the image sensor and the contrast unit in an axial rotation direction being fixed;
rotating the image wire that is positioned in a hollow organ so that an orientation of a distal end of the contrast unit changes in the tomographic images and the angiographic images on the display; and
determining the orientation of a distal end portion of the flexible body portion by viewing a change in the orientation of the distal end of the contrast unit in the tomographic images and the angiographic images displayed on the display.

18. The method according to claim 17, wherein the contrast unit is curved as seen in a side view.
